(19) United States
(12) Patent Application Publication
(10) Pub. No.: US 2009/0082758 A1
(43) Pub. Date: Mar. 26, 2009

(54) Implantable Fluid Distribution Device and Method of Fluid Delivery

(75) Inventors: Steven Streatfield Gill, Bristol (GB); Mathew David Frederick Stratton, Stroud (GB); Hugo George Derrick, Bristol (GB)

Correspondence Address: OLIFF & BERRIDGE, PLC, P.O. BOX 320850, ALEXANDRIA, VA, 22320-4850, US

(73) Assignee: REINSHAW PLC, GLOUCESTERSHIRE (GB)

(21) Appl. No.: 12/223,617

(22) PCT Filed: Feb. 13, 2007
(86) PCT No.: PCT/GB2007/000496

§ 371 Date: Aug. 5, 2008

(30) Foreign Application Priority Data
Feb. 15, 2006 (GB) 0603037.3

(51) Int. Cl. A61M 1/00

(52) U.S. Cl. 604/891.1

(57) ABSTRACT

An implantable fluid distribution device is described. The device includes a first inlet for receiving fluid under pressure from a remote pump. A plurality of outlets are also provided, each outlet being connectable to a fluid delivery catheter. A valve mechanism is arranged to control the passage of fluid from the first inlet to the plurality of outlets. Apparatus including the fluid distribution device, an implantable pump and a plurality of drug delivery catheters is also described. The fluid distribution device can be used to delivery drug to multiple regions in the body and the sequential delivery of drugs to different sites in the brain is disclosed.

The present invention relates to implantable apparatus for delivering fluids, such as drugs, to different parts of the human or animal body. In particular, the invention relates to an implantable drug distribution device for use with an associated drug delivery pump and a method of delivering drugs to the brain.

A variety of implantable drug delivery systems are known and typically comprise a drug pump assembly that can be implanted in the abdomen and one or more flexible catheters that route drug from the pump to the required anatomical site or sites. A drug reservoir may be integrated within the pump assembly or may be provided remotely to the pump. Typically, the drug reservoir or pump is designed for implantation close to the patient's skin so that it can be recharged percutaneously. Examples of known drug delivery pumps are described in U.S. Pat. No. 3,951,147, U.S. Pat. No. 4,013,074 and U.S. Pat. No. 4,692,147.

Although routing a drug to one anatomical site is sufficient for the treatment of certain medical conditions, there is often a requirement to deliver a drug to multiple sites within the body. For example, U.S. Pat. No. 5,752,930 describes drug delivery apparatus that comprises a catheter having a plurality of fluid exits. Similarly, US2004/0220545 describes the use of branched and/or multi-lumen catheters to route drugs from a pump assembly to different parts of the spinal column. Although the use of branched catheters or multiple aperture catheters permits drug delivery to different sites within the body, the relative flow of drug from the different exits of the catheter can be unpredictable. In particular, a catheter exit can be become obstructed or blocked which may result in reduced, or no, drug flow to certain sites whilst increasing the amount of drug delivered to other sites. This can seriously degrade treatment effectiveness.

WO2005/105199 describes a branched catheter systems that includes multiple fluid control valves, flow rate sensors and pressure sensors. In particular, WO2005/105199 outlines various methods of opening and shutting valves whilst monitoring pressure and fluid flow rate in different parts of the system. This is stated to allow any fluid flow problems (e.g. blockages or leaks) within the system to be found. The system of WO2005/105199 is, however, relatively complex to implement and implant within a subject.

WO2004/105839 describes a further implantable drug delivery pump that allows an accurate dosage of drug to be pumped from a reservoir to each of multiple sites within the body. The pump operates by pressing a rotor against two or more lengths of tubing to urge the drug therethrough. Separate lengths of tubing are then used to carry the drug from the abdominally implanted pump to each catheter. If required, the internal diameter of the lengths of tubing within the pump can be selected to control the relative flow of drug to each catheter.

According to a first aspect of the invention, an implantable fluid distribution device comprises; a first inlet for receiving fluid under pressure from a remote pump; a plurality of outlets, each outlet being connectable to a fluid delivery catheter; and a valve mechanism for controlling the passage of fluid from the first inlet to the plurality of outlets.

The present invention thus provides a fluid distribution device that can be implanted in a human or animal body and has a first inlet to which a pressurised supply of fluid can be fed from a remote pump. The device also has a plurality of outlets which may be connected to associated catheters as described in more detail below. The valve mechanism of the device of the present invention allows fluid communication between the first inlet and the plurality of outlets to be controlled as required. As described in more detail below, the valve mechanism is preferably a single N-port (where N is an integer value of at least three) flow control valve that can be switched between different configurations thereby providing, during use, active control over the flow of fluid from the first inlet to the plurality of outlets of the device. Although the device may be used to route any type of fluid, it is particularly suited to routing drug solutions and the like.

A fluid distribution device of the present invention allows drug delivery apparatus to be provided that has a number of advantages over known drug delivery systems. For example, the fluid distribution device can be relatively small compared with a pump assembly thereby allowing it to be implanted in a region that is much closer to the site(s) where the drug is to be delivered. In the case of a system for the delivery of drug to the brain, the fluid distribution device may be mounted on the patients skull only a few centimetres away from implanted catheters. This arrangement provides improved fluid flow control compared to systems, such as the pump described in WO2004/105839, where each catheter is connected to a pump implanted in the torso of a patient via a long length of tubing. Such an arrangement can also reduce stagnation effects that may occur after certain drugs are released from the pump assembly.

A fluid distribution device of the present invention also has a number of benefits compared with the distributed branched catheter system of WO2005/105199. In particular, the device of the present invention allows the entire fluid routing function to be provided if required by a single valve mechanism. In particular, a device of the present invention does not require a distributed network of two-port valves, flow restrictors, pressure sensors and various pieces of interconnecting tubing to be implanted in different parts of the body. A fluid distribution device of the present invention receives fluid from a remote pump and can distribute that fluid in the required manner to the plurality of outlets. It can thus be seen that the possibility of damage to, or failure of, the fluid distribution means is lower for an integrated device of the present invention compared to the networked system of WO2005/105199.

Advantageously, the device of the present invention comprises fluid flow control means for controlling the fluid flow rate through the device. The fluid flow control means may conveniently comprise one or more flow regulators located between the first inlet and at least one of the plurality of outlets. Preferably, any such discrete flow regulators are located within the housing of the fluid distribution device. Each flow regulator may have fixed properties (e.g. it may comprise a length of narrow diameter tubing) or may have variable properties (e.g. it may be arranged to provide a variable constriction in the fluid pathway between the inlet and outlet). The fluid flow control means may comprise a fixed flow regulator connected in series with a variable flow regulator; for example, the fixed flow regulator may reduce the pressure by a given amount and the variable flow regulator could then “fine tune” the flow rate to the required level.

Although discrete flow regulators may be provided, in a preferred embodiment the valve mechanism itself may comprise integral fluid flow control means to control the flow rate of fluid passing therethrough. The valve mechanism may thus provide “on”, “off” and one or more intermediate flow rate states. In other words, the valve mechanism may perform both a fluid routing and a flow control function. Providing a single valve mechanism for both selectively routing fluid from the first inlet to the desired outlet and also controlling the flow rate of any such routed fluid enables a more compact device to be provided. In particular, such a device would not require a complex network of distributed flow restrictors and valves of the type described in wo2005/105199.

The provision of flow control as described above enables fluid to be expelled from an outlet at a lower pressure than it is received at the first inlet. This allows, for example, fluid to be supplied to the fluid distribution device at a relatively high pressure whilst fluid is routed to a catheter at a lower pressure. If variable flow regulators are provided, the pressure of fluid supplied to each catheter may be dynamically, possibly continuously, adjusted as required to optimise fluid delivery. It should be noted that the fluid flow rate to a given outlet, and the pressure of fluid at that outlet, will be interdependent for a given device. In other words, reducing the fluid flow to an outlet can also reduce the pressure of fluid at that outlet for a given system set-up.

Conveniently, the valve mechanism provides a shut-off state in which no fluid can pass from the first inlet to any of the plurality of outlets. In other words, the valve mechanism may be configured to have a fully closed state in which fluid communication is prevented between the first inlet and all of the outlets. The ability to stop any fluid flow through the device enables fluid delivery to be interrupted as and when required. This may be advantageous when bolus delivery is required; e.g. when delivery of a drug at a high flow rate is to be interspersed with periods of no drug delivery.

Advantageously, the device comprises two or more sets of outlets, each set of outlets comprising at least one of said plurality of outlets, wherein the valve mechanism permits fluid to pass from the first inlet to no more than one set of outlets at a single instant in time. The sets of outlets may comprise common outlets; i.e. they may be non-exclusive sets. However, the sets of outlets are advantageously mutually exclusive. In this manner, the valve mechanism can route fluid to only some of the outlets at a single time. As described below, such an arrangement can allow fluid to be routed to the sets of outlets in a sequential manner if required. Each set may comprise a single outlet, in which case fluid communication can be established, as required, between the first inlet and any selected one (and only one) of said plurality of outlets in turn.

Preferably, the device comprises a controller for controlling operation of the valve mechanism. The controller may be an electronic device or an electro-mechanical device. The controller is preferably arranged to apply appropriate control signals to the valve mechanism so that it adopts the required state. Conveniently, the controller is programmable; i.e. the controller may store a set of instructions for controlling operation of the valve mechanism. Alternatively, the controller may be arranged to receive a control signal from a remote interface and to adjust the configuration of the valve mechanism accordingly. It should be noted that although providing a controller to control operation of the valve mechanism is preferred, such a component is not essential. The valve mechanism could, if desired, be manually re-configurable. For example, the valve mechanism may be capable of manual readjustment by a user, possibly through the skin after implantation.

The controller can advantageously be programmed with a set of instructions for controlling the valve mechanism in the desired manner prior to implantation within a patient. Advantageously, the controller can be programmed over a communications link. For example, the controller may be programmed via a wireless (e.g. inductive, RF etc) communications link. The ability to programme, or re-programme, the controller remotely allows the fluid delivery process to be altered before, during or after implantation of the device within the body. This can be highly advantageously if the drug delivery profile requires modification after implantation of the device. The controller may also be programmed with instructions for priming the device during implantation. For example, the device may be arranged to provide maximum fluid flow for a set period or until it receives an instruction (e.g. via the communications link) that implantation has occurred and that the required drug delivery programme should be initiated.

Advantageously, the device comprises two or more sets of outlets, each set of outlets comprising at least one of said plurality of outlets, wherein the controller is arranged to operate the valve mechanism such that fluid is passed from the first inlet to each set of outlets in sequence. The sets may be non-exclusive (e.g. an outlet may be included in more than one set) or mutually exclusive. It should be noted that the valve mechanism may allow for simultaneous fluid output to each outlet, but the controller may be arranged such that fluid is passed to sets of outlets in turn (e.g. to provide sequential fluid delivery). Alternatively, the valve mechanism may take the form described above and only permit fluid to be routed to one set of outlets at a time. In a preferred embodiment, the controller may be programmed to reconfigure the valve mechanism so that fluid communication between the first inlet and each of the sets of outlets is sequentially established.

The controller advantageously comprises a clock thereby allowing the valve mechanism to be automatically adjusted at predefined time intervals. In this manner, switching fluid flow between each outlet or set of outlets may be timed. For example, the controller may be arranged to route fluid through each outlet, or each set of outlets, in turn for a preset period of time. Furthermore, the controller may be programmed to alter the flow rate of fluid through the different outlets, or the pressure of fluid at an outlet, over time. If a timed arrangement is implemented, the clock of the controller may be synchronised with the clock of a remote electrical pump. For example, the remote pump may vary the pressure under which fluid is expelled to coincide with the fluid distribution device routing such fluid to different catheters. Also, the pump may be stopped during intervals when no fluid is being passed through the fluid distribution device.

Instead of timed operation, the controller may reconfigure the valve mechanism to direct fluid to another outlet or set of outlets when a certain condition is met. For example, the outlet may be switched in response to reduced fluid pressure at the first inlet (e.g. if the remote pump is programmed to provide fluid at different pressures during different time intervals) or when a certain amount of fluid (e.g. a certain drug dosage) has passed through an outlet. The controller may also be operated directly by a remote unit; e.g. over a wireless link. For example, instructions to alter the valve mechanism may be received from a remote unit such as a user interface or the remote pump. A single master controller may also be provided to control both the fluid distribution device and the remote pump.

Providing a fluid distribution device that can selectively route fluid to different outlets has a number of advantages, especially if sequential delivery is automatically implemented under the control of the controller. In particular, it enables drug delivery to be rotated through a number of different sites with the body (e.g. within the brain). For example, such sequential delivery would allow a chemotherapy drug to be delivered to the whole brain over a certain period. Such sequential delivery would remove the need to simultaneously pump large volumes of chemotherapy drug into the whole brain via multiple catheters thereby reducing the side-effects associated with the chemotherapy treatment without reducing the effectiveness of such treatment. Reducing the maximum required flow rate also reduces the cost and size of the associated pump assembly.

In order to monitor fluid delivery, the device of the present invention advantageously comprises at least one sensor for sensing at least one of fluid pressure and fluid flow rate. In other words, the device may include integral fluid pressure sensing means and/or fluid flow rate sensing means. Conveniently, the at least one sensor may be located within the valve mechanism.

The at least one sensor may advantageously comprise at least one pressure sensor. A pressure sensor may be provided to measure the pressure of fluid received at the inlet and/or expelled via one or more of the outlets. Each pressure sensor may comprise, for example, a silicon strain gauge. Data from the pressure sensor(s) may be recorded or output from the device, e.g. via a telemetry link, as required.

The at least one sensor may conveniently comprise one or more fluid flow rate sensors. A flow rate sensor may be provided to measure the rate at which fluid flows into the device via the first inlet and/or the rate at which fluid is output via one or more of the outlets. It should be noted that fluid flow rate may also be measured using a plurality of pressure sensors; the pressure drop between two points along the fluidic path providing a measure of flow rate. Measurement of fluid flow rate enables the amount of fluid received by the fluid distribution device and/or the amount of fluid dispensed via an outlet to be determined, thereby allowing drug dosage to be measured. Data from the flow rate sensor(s) may be recorded or output from the device, e.g. via a telemetry link, as required.

If the device comprises at least one sensor as described above, the controller may be arranged to receive the fluid pressure and/or fluid flow rate data. For example, the controller may receive fluid pressure data from pressure sensor(s) and fluid flow rate data from flow rate sensor(s). The controller may be arranged to store such data and/or forward such data to a remote interface. Data from the at least one sensor may be used by the controller for controlling operation of the device; e.g. enabling a feedback control loop to be established. In one embodiment, the controller may be programmed to control the valve mechanism in response to measured flow rate and/or fluid pressure data. For example, the outlet to which fluid is passed can be altered when a certain amount of fluid has been passed through that outlet. Fluid delivery could also be prevented altogether when a certain amount of fluid has been dispensed in a set period of time.

The controller conveniently comprises a memory portion. This may be used to store historical fluid delivery data. For example, it may store drug delivery data as a function of time and/or the outlet through which the drug was delivered. Such data may be downloadable to a remote unit via a communications link. In one example, the communications links may comprise a Bluetooth connection. Data may then be sent from the device to a remote computer interface (e.g. at a hospital) via a Bluetooth compatible mobile telephone or similar.

The controller advantageously comprises an alarm. The alarm may be arranged to emit a sound (e.g. a beep) or other indication when a fault or other problem with operation of the device is identified. In other words, the controller may be arranged to perform a self monitoring function. The alarm may be sounded, for example, when fluid pressure strays outside a predetermined range, when fluid flow increases or decreases beyond certain limits or if the battery is running low. The controller may also be arranged to place the device into a “safe” configuration in the event of an alarm. For example, the device may be switched into an “off” state in which all fluid flow is stopped if the battery is running low or if an increased and unwanted increase in fluid flow rate is detected. A signal may also be sent to a remote interface in the event of an alarm condition (e.g. via a Bluetooth link).

Although only a first inlet may be provided, the device advantageously comprises at least one additional inlet. In other words, the device may comprise a plurality of inlets. Conveniently, the valve mechanism also controls the passage of fluid from the at least one additional inlet to the plurality of outlets. Each inlet may be arranged to receive fluid under pressure from a remote pump or pumps. Each inlet may receive a different fluid and the fluid distribution device may comprise some sort of chamber or other region for mixing such fluids. Advantageously, the valve mechanism comprises a mixing chamber for receiving fluid from the first inlet and from the at least one additional inlet. The mixture ratio may be controlled as required allowing different mixtures (e.g. different drug combinations) to be routed to different outlets or sets of outlets. The mixture ratio may also vary with time. Each inlet may, as desired, comprise a length of flexible tubing permanently attached thereto.

Although the device may comprise only two outlets, more outlets may be provided.

The device may thus conveniently comprise three or more, four or more, five or more, six or more, seven or more, eight or more, or ten or more outlets. It should be noted that a device may be provided that has more outlets than will always be required. For example, a device having eight outlets may be used in applications where fewer than eight outlets are actually required. If fewer outlets are required, cap(s) may be used to seal any unused outlets to prevent the accidental release of fluid. The outlets may be marked (e.g. numbered or colour coded) to allow them to be readily identified by a surgeon during implantation.

In addition to the plurality of outlets that are connectable to fluid delivery catheters, the device may advantageously comprise one or more fluid return outlets to which fluid received at an inlet may be routed. The device may thus be configured to provide one or more flow return paths that allows fluid to be routed back to the remote pump. Such a flow return outlet may be used for pressure release or bleeding purposes and may also be used to prevent fluid stagnation effects within the device.

Fluid may be routed back to the pump from the device via a length of tubing that is separate to the tubing through which fluid is supplied from the pump or multi-lumen tubing may be used to separately route fluid to and from the associated pump.

Advantageously, the valve mechanism comprises a chamber, wherein the chamber is in fluid communication with the first inlet and has a plurality of output apertures, wherein any fluid exiting the chamber of the valve assembly via an output aperture is routed to one of said plurality of outlets. Conveniently, the valve mechanism comprises a moveable plug associated with each output aperture, wherein movement of each moveable plug controls fluid flow through the associated aperture. Each moveable plug may comprise a resiliently deformable portion (e.g. a rubberised end cap) for engaging its associated aperture. Instead of each plug directly engaging its aperture, a flexible diaphragm may be located between the plugs and apertures. In such an example, the plug may act to force the diaphragm into contact with the aperture to provide the fluid flow control.

The valve mechanism may conveniently comprise a plurality of electrically powered actuators, each electrically power actuator being arranged to move at least one of said moveable plugs. In one embodiment, each moveable plug may comprise an associated electrically powered actuator. Each electrically powered actuator may be bistable thereby enabling each plug to be held in either the “on” or “off” state without having to expend any power. Alternatively, the valve mechanism may conveniently comprise a single moveable member that can urge each of said plurality of moveable plugs into contact with its associated aperture. The valve mechanism may thus conveniently comprise a moveable member (e.g. a rotatable disc having a recess or protrusion formed therein) for selectively urging any one or more of said plurality of moveable plugs into contact, or out of contact, with the associated aperture. Each moveable plug may be spring loaded to return to a predetermined location in the absence of an applied force. For example, the moveable plugs may be spring loaded so as to return to an “open” or “closed” position when no force is applied by the moveable member.

Although the above described valve assembly can advantageously form part of an implantable fluid distribution device, it may also be used in other (e.g. non-medical) applications. A valve assembly may thus be provided that comprises a chamber in fluid communication with an inlet and having one or more output apertures, the valve assembly also comprising a moveable plug associated with each output aperture, wherein movement of the moveable plug relative to the aperture controls fluid flow therethrough. Fluid may exit the chamber via any selected aperture by appropriately moving the moveable plugs in the manner described above.

Conveniently, the valve mechanism comprises an inner member substantially retained within, and moveable relative to, an outer member. Advantageously, the inner member encloses or defines a cavity in fluid communication with said first inlet. The inner member may also or alternatively comprise one or more channels in fluid communication with the first inlet. The outer member of the valve assembly preferably comprises a plurality of output ports, each output port being in fluid communication with one of said outlets. The inner member conveniently has at least one aperture formed therein. Movement of the inner member relative to the outer member advantageously permits fluid to be selectively routed from the first inlet to any of said outlets through the at least one aperture of the moveable member. Such a valve mechanism may comprise a single aperture that can be aligned with any of the output ports or a plurality of apertures, wherein each aperture can be aligned with one associated output port. The inner member may be moveable relative to the outer member in a number of ways. Advantageously, the inner member may be axially translatable relative to the outer member. Conveniently, the inner member is rotatable relative to the outer member. Axial translation, or rotation, of the inner member thus controls the fluid path through the device accordingly.

A valve assembly or mechanism is thus described herein, the valve comprising an inlet, an outer portion comprising one or more outlets, and an inner portion that is moveable relative to the outer portion, wherein movement of the inner portion relative to the outer portion allows fluid communication to be selectively established between the first inlet and any one or more of the outlets. The inner portion may have at least one aperture formed therein and may define a cavity that is in fluid communication with the inlet. The inner portion may be movable into any one of a plurality of positions such that fluid can be selectively routed from the inlet to any one or more of the plurality of outlet via the at least one aperture of the moveable member. Such a valve may be used in a fluid distribution device as described above or may be used in any one of a variety of alternative (e.g. non-medical) devices.

Advantageously, the valve mechanism of the device of the present invention comprises a first length of resiliently deformable tubing for receiving fluid from the first inlet and one or more additional lengths of resiliently deformable tubing for delivering fluid to the plurality of outlets. The valve mechanism may then advantageously comprise a single flow control member that can be selectively urged against each of the one or more additional lengths of resiliently deformable tubing thereby controlling the passage of fluid therethrough. Although an on/off arrangement may be provided, the flow control member advantageously provides variable deformation of each associated length of resiliently deformable tubing thereby controlling the rate of fluid flow therethrough. The valve mechanism may comprise an electrically powered actuator for moving said flow control member. The actuator may, for example, comprise a stepper motor and/or a piezo-electric actuator. A valve mechanism in which a piece of flexible tube is deformed has the advantage of not requiring valve components to be located in the fluidic path. Such devices are also likely to be less susceptible to mechanical wear that may lead to fluid leakage.

The flow control member may comprise one or more segments, each segment being located adjacent one of the pieces of resiliently deformable tubing that leads to an outlet. Each segment of the flow control member may have a cross-sectional profile comprising a circular portion and a substantially flat portion. Rotation of the flow control member may then bring the flat or circular cross-sectional portion of each segment into contact with the associated resiliently deformable tubing as required. The circular cross-section portion of each segment can be arranged to deform the associated flexible tube thereby preventing fluid flow through that tube and the substantially flat cross-section of the segment can be arranged to impart reduced (or zero) tube deformation thereby allowing fluid to pass through the tube. The flat portions of each segment may be offset so that, at any one time, fluid may pass through only one of the plurality of resiliently deformable tube. Rotation of the flow control member thus allows the tube which is “open” to be selected as required and may also be used to control the rate of fluid flow through the selected “open” tube.

A valve or fluid routing means is thus described herein that comprises resiliently deformable tubing (e.g. tubing comprising plastic) and a moveable flow control member, wherein the moveable flow control member can be urged against said resiliently deformable tubing to restrict the flow of fluid therethrough. The valve may comprise a first length of resiliently deformable tubing for receiving fluid from the first inlet and one or more additional lengths of resiliently deformable tubing for delivering fluid to one or more outlets, wherein the flow control member can be urged against any one or more of said first and additional lengths of resiliently deformable tubing. A fluid routing means is thus provided that comprises a plurality of deformable tubes for carrying fluid and flow control means that can be selectively urged against any one or more of said plurality of resiliently deformable tubes to restrict fluid flow therethrough. Such a fluid control device may provide a valve mechanism of the present invention or may be used in any number of alternative (e.g. non-medical) applications.

Advantageously, the device comprises an integral electrical power source for powering at least one electrically powered component (e.g. for powering the valve mechanism). Alternatively, the device may comprise an electrically powered component and means for receiving electrical power from a remote power source. For example, an electrical cable may be routed to the device from a remote, implanted, battery. Preferably, any such remotely located battery is contained in the remote pump assembly. Although the power source is preferably a battery, it may alternatively comprise a kinetic power source or a RF harvesting power source. Means may also be provided for coupling external power into the device (e.g. to recharge a battery) through the skin; e.g. the device may comprise an inductive power coupling.

Preferably, the first inlet and each of the plurality of outlets comprise tube connection means. For example, the connection means may comprise a nozzle to which tubing can be attached with a suture or a snap fit connector. Alternatively, one or more lengths of tubing may be permanently connected to the device (e.g. connected during device manufacture). For example, the inlet may comprise a piece of flexible tubing (e.g. tubing comprising plastic) that is sufficiently long to run to the remote pump. The end of the tubing that is to be connected to the remote pump may comprise a integral connector, such as a screw thread, that can be securely attached to a complementary connector on the remote pump. Similarly, each outlet may comprise a length of flexible tubing that is sufficiently long to reach the desired anatomical site and is permanently attached to the device. If more than one outlet is provided, each outlet may comprise a similar length of permanently attached flexible tubing. Providing permanent fluid connections reduces the risk of leakage from the device during, or after, implantation and also decreases the time required to implant the device within a patient.

The device may conveniently comprise one or more bacterial filters. For example, at least one or all of the outlets may include a bacterial filter. A bacterial filter may also, or alternatively, be provided at said at least one inlet. Furthermore, one or more bacterial filters may be included within the valve mechanism itself. Providing appropriately located bacterial filters ensures that no bacteria are transported through and expelled from the catheters connected to the device. This is especially important when fluids are being delivered directly into the brain through the blood-brain barrier.

One potential use of a device of the present invention is to deliver viral, e.g. gene therapy, material to regions of the body. It is thus preferred that substantially all of the fluid contacting surfaces of the valve mechanism are formed from a material that does not significantly reduce viral activity (e.g. of gene therapy viral vectors). The device may thus advantageously comprise fluid contacting surfaces that comprise at least one of polypropylene, High Density Polyethylene (HDPE), Polytetrafluoroethylene (PTFE), Ethylene/Tetrafluoroethylene Copolymer (ETFE), Flouroethylene-propylene (FEP), Polyethylene Terephthalate (PET) and polyurethane. The compatibility of such materials with the delivery of viral vectors is described in MOLECULAR THERAPY Vol. 1, No. 5, May 2000, Part 1. The fluid contacting surfaces may also conveniently comprise glass (e.g. fused silica, silica, quartz etc) or ceramic (e.g. zirconia). Any combination of such materials may be provided as required.

Advantageously, the fluid distribution device is adapted to be mounted subcutaneously; i.e. the device is implantable under the skin. Preferably, the device is adapted to be mounted, at least in part, in a recess or hole that is formed in the skull. The device may have one or more flanges protruding therefrom which can be attached to the skull (e.g. using screws). Conveniently, the device comprises an outer, substantially cylindrical, housing. The device may have a domed upper surface; i.e. so that there are no sharp edges which may cause a site of infection. The inlet(s) and outlets of the device may protrude radially from the housing. The housing may be formed from Titanium. Alternatively, the device and/or the components contained therein may be formed from a plastic or other non-magnetic material so that a patient having a device implanted therein may have a magnetic resonance imaging (MRI) scan.

Advantageously, the device is formed using one or more micro-electro-mechanical system (MEMS) components. For example, the device may comprise MEMS based actuators, pressure sensors (e.g. Silicon strain gauges) etc.

In accordance with the present invention, implantable drug delivery apparatus may be provided that comprises an implantable fluid distribution device as described above. The apparatus preferably also comprises at least one pump assembly. The pump assembly may be implantable and may comprise antibacterial filters and the like. A length of flexible, implantable, tubing may be advantageously provided to carry fluid from a pump assembly to the first inlet of the fluid distribution device. If the fluid distribution device comprises a plurality of inlets, a plurality of tubes (or a multi-lumen tube) may be provided to link a pump to each inlet. The tubing may comprise plastic (e.g. polyurethane). Such apparatus allows the delivery of drugs from one or more remote pumps, via the fluid distribution device, to one or more different sites within the body.

As described above, the flexible tubing that links the pump to the fluid distribution device may have a first end that is permanently attached to the fluid distribution device and a second end that comprises a connector. The pump assembly may then comprise a complimentary connector (e.g. a port having an external screw thread) suitable for attachment to the connector carried by the second end of the flexible tubing. In this manner, the tubing can be securely attached to both the pump assembly and the fluid distribution device. The pump assembly may also have a self-closing valve that is arranged to prevent fluid egress from the pump assembly when the associated flexible tubing is not securely attached. For example, the connector on the pump may be a self-closing connector. Alternatively, the pump assembly may be arranged to measure fluid flow or output fluid pressure and to stop the supply of the fluid if the flow rate increases, or the pressure drops, to such an extent that a leak is likely to have occurred.

Advantageously, the pump assembly comprises a constant pressure pump. The pump assembly may include one or more reservoirs for storing a fluid containing a drug or drugs. The pump may be implanted in any suitable location within the body; e.g. the pump may be adapted for abdominal implantation. The pump assembly, including any reservoir, may have means for percuteneously refilling the fluid reservoir. Conveniently, a length of flexible tubing is provided to carry fluid from the pump to the fluid distribution device. The tubing may be tunnelled subcutaneously. The tubing may be longer than 10 cm, longer than 20 cm, longer than 50 cm, or longer than 1 m. The tubing may be sufficiently long to enable a coil of tubing to be provided within the patient to allow for movement.

If the pump assembly comprises a power source for powering the fluid distribution device and/or control electronics for controlling the function of the fluid distribution device, the tubing may comprise a plurality of conductive tracks running along its length. These conductive tracks or wires are preferably electrically insulated from one another and are arranged to carry the necessary signals and/or electrical power between the pump assembly and the fluid distribution device. The electrical wires may be wrapped around the tubing or the tubing may comprise such wires embedded in a outer (e.g. plastic) wall. The wall in which the wires are embedded may form the internal wall of the tubing. The tubing may comprise a connector on one or both ends (e.g. the end that connects to the pump assembly and/or the fluid distribution device) that provides both a fluid and electrical connection. Alternatively, separate fluid and electrical connectors may be provided as required. If a communications link between the pump and fluid distribution device is established, a single controller may be provided to control operation of both the pump and the fluid distribution device.

Implantable tubing is thus described herein that comprises a wall defining a core or lumen for carrying a fluid, wherein the wall also comprises one or more conductive tracks. The conductive tracks or wires may advantageously be co-axial with the fluid carrying core. Connectors may be provided on one or both ends of the tubing as required. The connector(s) may provide both a fluid and electrical connection to a complementary connector (e.g. a connector on a pump). Such tubing may be used in apparatus of the type described above and may also be used in various different applications.

Advantageously, the apparatus comprises a plurality of catheters, wherein each outlet of the fluid distribution device is connectable or connected to a fluid delivery catheter. Each outlet of the fluid distribution device may be connected to a fluid delivery catheter via a length of flexible tubing. The apparatus may thus comprise flexible tubing to carry fluid from the outlets of the fluid distribution device to the catheters; any such tubing may be tunnelled subcutaneously across the scalp. If delivery to the brain of a subject is required, the catheters may be of the type described in WO03/077785.

As mentioned above, the fluid distribution device may include one or more sensors for sensing fluid pressure and/or fluid flow rate therein. One or more sensors for sensing the pressure and/or flow rate of fluid may be conveniently provided at one or more locations within the apparatus (e.g. at the pump or a catheter). Some, or all, of these sensors may be provided outside the fluid distribution device. If a controller for controlling operation of the valve mechanism of the fluid distribution device is provided, that controller may also be arranged to monitor the fluid pressure (and/or flow rate) at locations external to the fluid distribution device. Advantageously, the controller is arranged to ensure that the fluid pressure within the apparatus does not exceed a predetermined value. As described above, the controller may issue a pressure warning signal if the predetermined pressure value is exceeded.

According to a further aspect of the invention, a method of surgery comprises the step of implanting one or more fluid distribution devices as described above. Conveniently, the method also comprises the step of implanting at least one of a pump assembly and flexible tubing. Advantageously, the method also comprises the step of implanting at least one catheter. Preferably, the step of implanting at least one catheter comprises the step of implanting at least one catheter that permits the delivery of fluid to one or more parts of the central nervous system. Preferably, the catheters are implanted to permit delivery of fluid to the brain. The location of such catheters would depend on the particular treatment that is required.

According to a yet further aspect of the invention, a method for delivering drugs to the brain comprises the steps of: (a) taking a subject brain having a plurality of catheters implanted therein, and (b) sequentially delivering a fluid to two or more mutually exclusive subsets of said catheters. Each subset of catheters may comprise one or more catheters. Advantageously, step (a) comprises implanting a plurality of catheters into the subject brain. Preferably, step (a) comprises implanting said plurality of catheters in locations that allow a drug to be delivered to substantially the whole brain.

The sequential delivery of drugs to different sites within the brain may improve treatment efficacy. For example, the delivery of a drug to the entire brain could be most efficiently achieved using seven catheters. For example, a first catheter pair (set A) could be inserted to deliver drug to the left and right frontal lobes. A second catheter pair (set B) could then be inserted to permit delivery to the left and right parieto-occipital lobes. A third catheter pair (set C) could be inserted into the left and right tempo-occipital lobes. A catheter (set D) could also be inserted into the pons to allow drugs to be driven down into the cerebellum through the white matter tracks.

The method could thus involve delivering a drug sequentially to the catheters of each of sets A, B, C and D. For example, drug could be pumped to the catheters of set A for an hour, followed by pumping drug to the catheters of set B for an hour, followed by pumping drug to the catheters of set C for an hour, followed by pumping drugs to the catheter of set D for an hour. The cycle could then be repeated as many times as required. The cycle could be interspersed with periods of no drug delivery and the time during which the drug is delivered may be different for the different sets of catheters as required. The sequence (i.e. A, B, C, D) could also be altered or may vary over time as required.

Advantageously, step (b) comprises determining the amount of fluid delivered to each catheter. For example, the flow rate may be measured or estimated. In such a case, the next step in the delivery sequence could be triggered when a certain amount of fluid has been delivered. Step (b) may thus comprise selecting the amount of fluid that is delivered to each catheter.

As noted above, step (b) may advantageously be repeated a plurality of times. For example, drug delivery may be performed for hours, days, weeks, months or years. Furthermore, the relative amount of fluid delivered by each catheter may be altered between repetitions of said step (b). In other words, the sequence and/or duration of drug delivery via each of the catheters may be varied over time. Such a variation in the drug delivery scheme may be required if, say, a neurotrophin is delivered. The neurotrophin may cause nerve growth or axonal sprouting at a desired target site within the brain. Once the growth/sprouting is established, drug may be delivered to a more discrete area where the nerve cell bodies are located which would maintain the integrity of the newly sprouted axons and nerve connections. In other words, the drug delivery profile can be adapted as the brain structure is altered by the drug it is receiving. Also, the drug delivery profile may be altered by a physician in light of the patient's response to the treatment.

Step (b) preferably comprises the step of delivering a drug. A number of drugs for treating a variety of conditions are described in more detail below. The fluid delivered may be a liquid having active components dissolved therein (e.g. drugs, dyes etc) or it may be a liquid containing small solid particles (e.g. viruses, viral vectors, liposomes, nanoparticles, gene therapy agents etc). Preferably, step (b) comprises delivering a fluid via convection enhanced delivery. In other words, the pressure of fluid output by the catheter is selected to provide convection enhanced delivery. The method may comprise the step of using apparatus of the present invention as described herein or apparatus of any other type.

An implantable fluid distribution device is thus described herein that comprises a first inlet for receiving fluid under pressure from a remote pump and one or more outlets, wherein the device comprises reconfigurable fluid routing means (e.g. a fluid routing valve or valve mechanism) that enables fluid communication to be selectively established between said first inlet and said one or more outlets.

A skull mountable fluid distribution device is also described herein that comprises at least one inlet and one or more outlets. The device may be adapted to be mounted, at least in part, in a recess or hole that is formed in the skull. The device may have one or more flanges protruding therefrom which can be attached to the skull (e.g. using screws). The device may be passive (e.g. have no moving parts) or may comprise active components such as fluid routing means (e.g. valve assemblies etc) of the type described above. The device may also comprise a pump and/or reservoir. The device may be formed from MEMS components and may have one dimension that is less than 5 cm, or more preferably less than 3 cm or more preferably less than 1 cm. The device is preferably suitable for mounting on a human skull. Preferably the device comprises a plurality of outlets.

An implantable fluid distribution device is also described herein that comprises a first inlet (e.g. for receiving fluid under pressure from a remote pump) and a plurality of outlets, said plurality of outlets being divided into two or more mutually exclusive sets of outlets, wherein the device comprises reconfigurable fluid routing means (e.g. a valve mechanism) that allows fluid communication to be selectively established between the first inlet and any one of said two or more mutually exclusive sets of outlets. Preferably, each mutually exclusive set of outlets comprises a pair of outlets. As described above, each outlet may be connected to a catheter. The device may be arranged to route fluid to each of the mutually exclusive set of outlets (and hence catheters) in turn. The device may also comprise a pump and/or reservoir. The device may be formed from MEMS components and may have one dimension that is less than 5 cm, or more preferably less than 3 cm or more preferably less than 1 cm. The device is preferably suitable for mounting on a skull.

The invention will now be described, by way of example only, with reference to the following drawings in which:

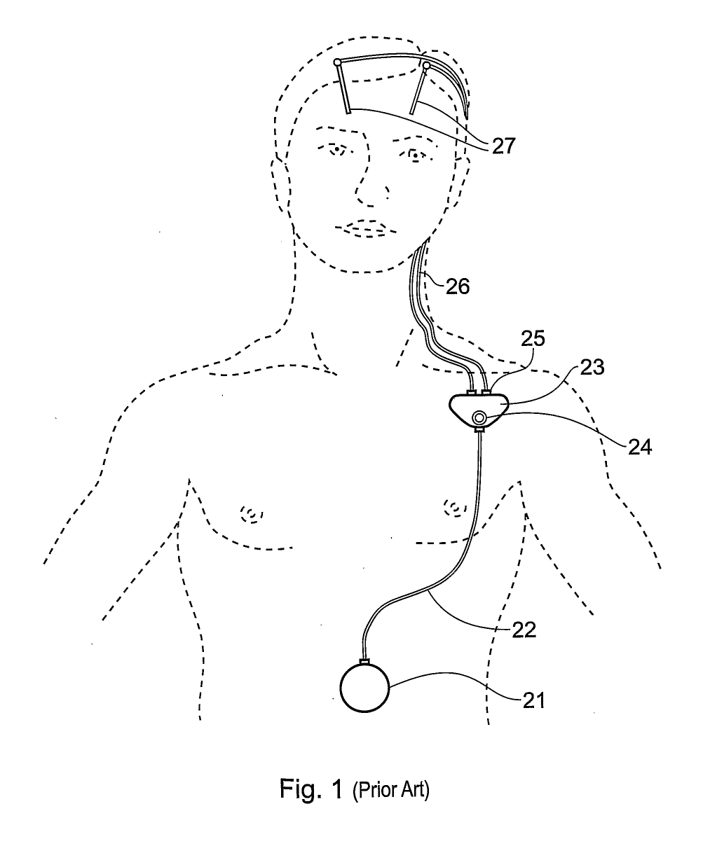

FIG. 1 illustrates a prior art drug delivery apparatus for simultaneously delivering drugs to different parts of the brain,

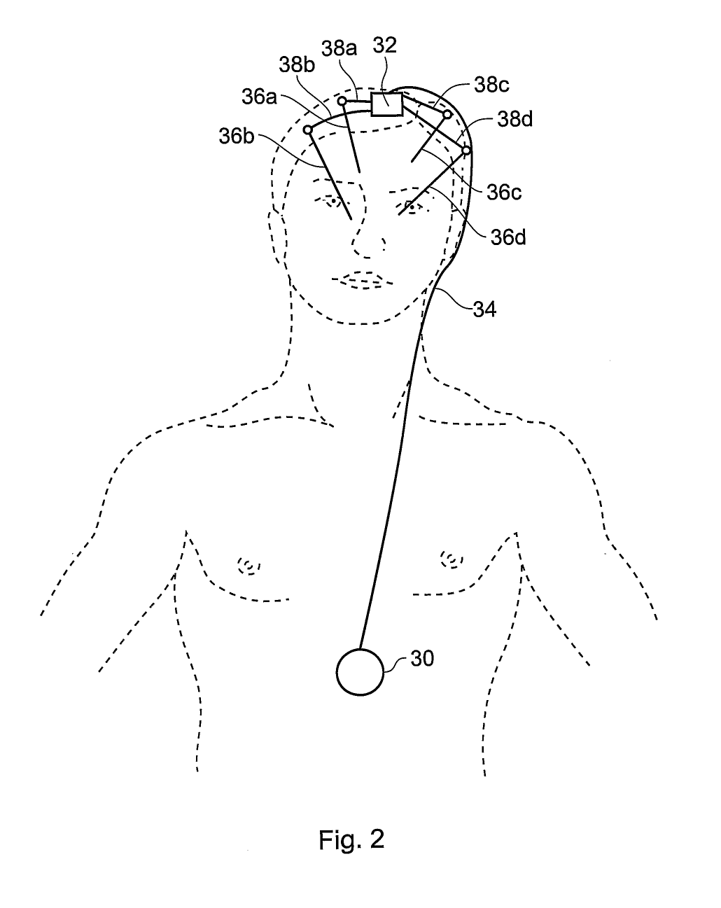

FIG. 2 illustrates drug delivery apparatus of the present invention for sequentially delivering drugs to different parts of the brain,

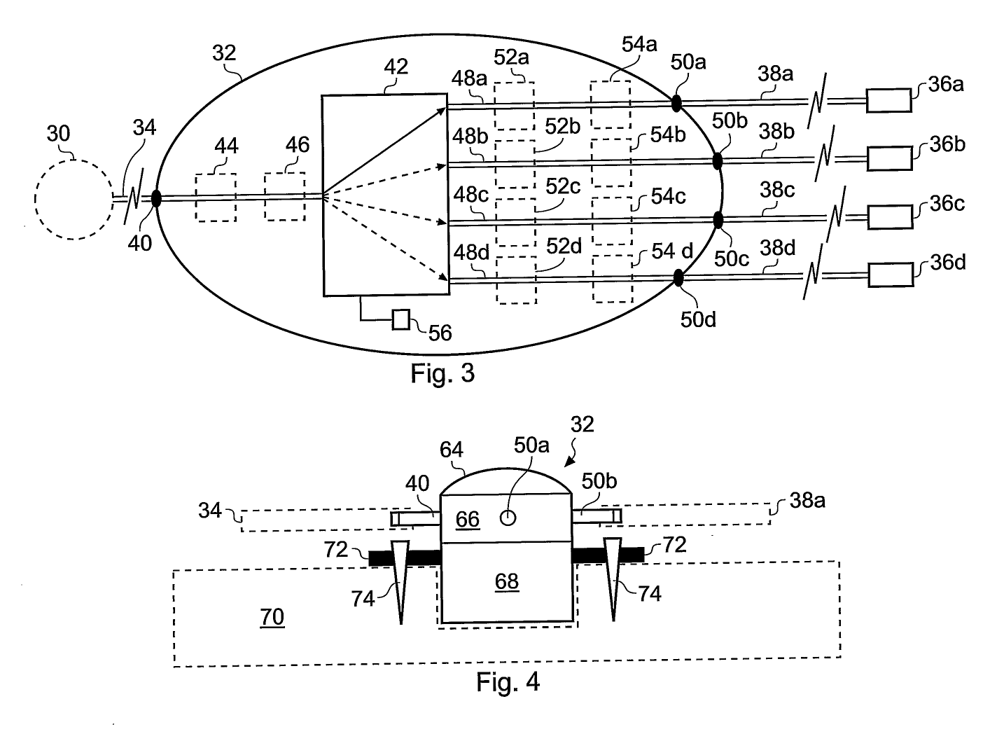

FIG. 3 shows the various components of the fluid distribution device illustrated in FIG. 2,

FIG. 4 shows the outer housing of the fluid distribution device shown in FIGS. 2 and 3,

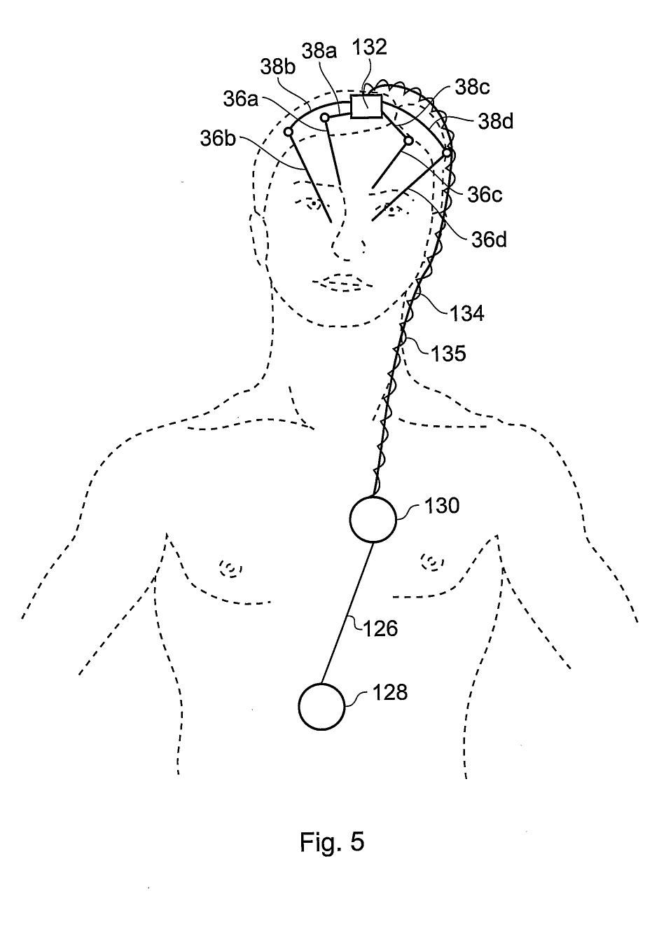

FIG. 5 shows an alternative drug delivery apparatus of the present invention for sequentially delivering drugs to different parts of the brain

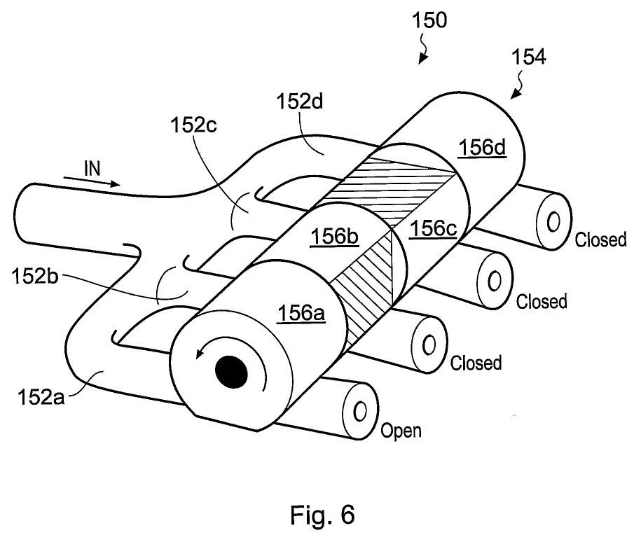

FIG. 6 shows a first valve assembly suitable for use as a fluid distribution device,

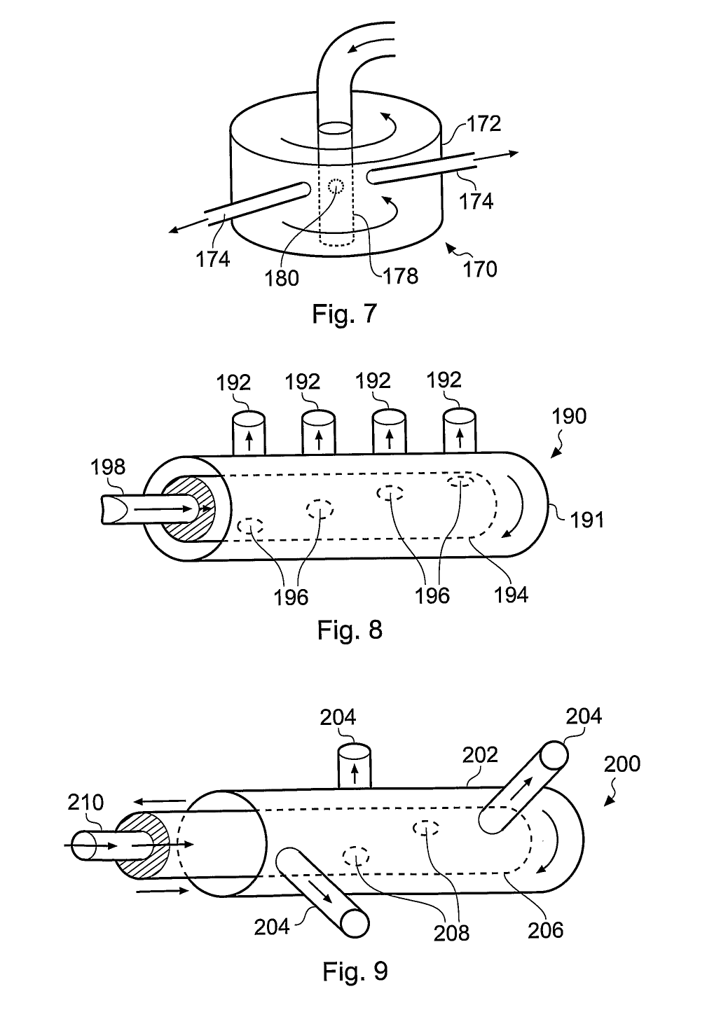

FIG. 7 shows a second valve assembly suitable for use as a fluid distribution device,

FIG. 8 shows a third valve assembly suitable for use as a fluid distribution device,

FIG. 9 shows a fourth valve assembly suitable for use as a fluid distribution device,

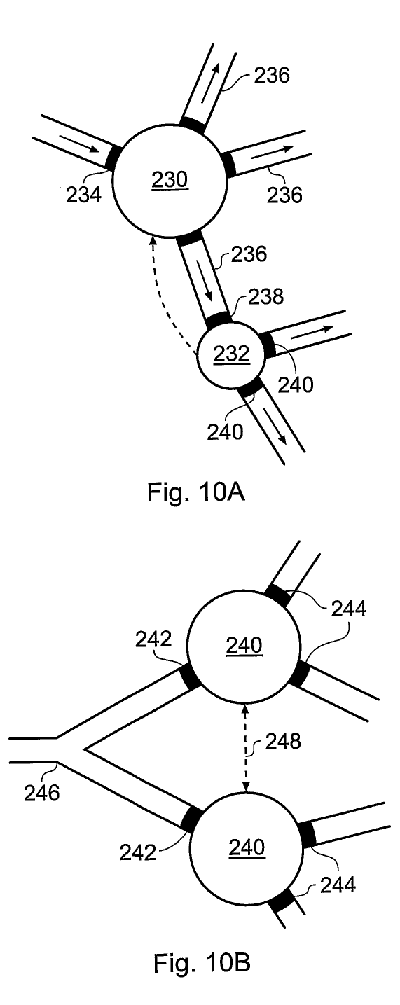

FIG. 10 illustrates the combination of a number of fluid distribution devices,

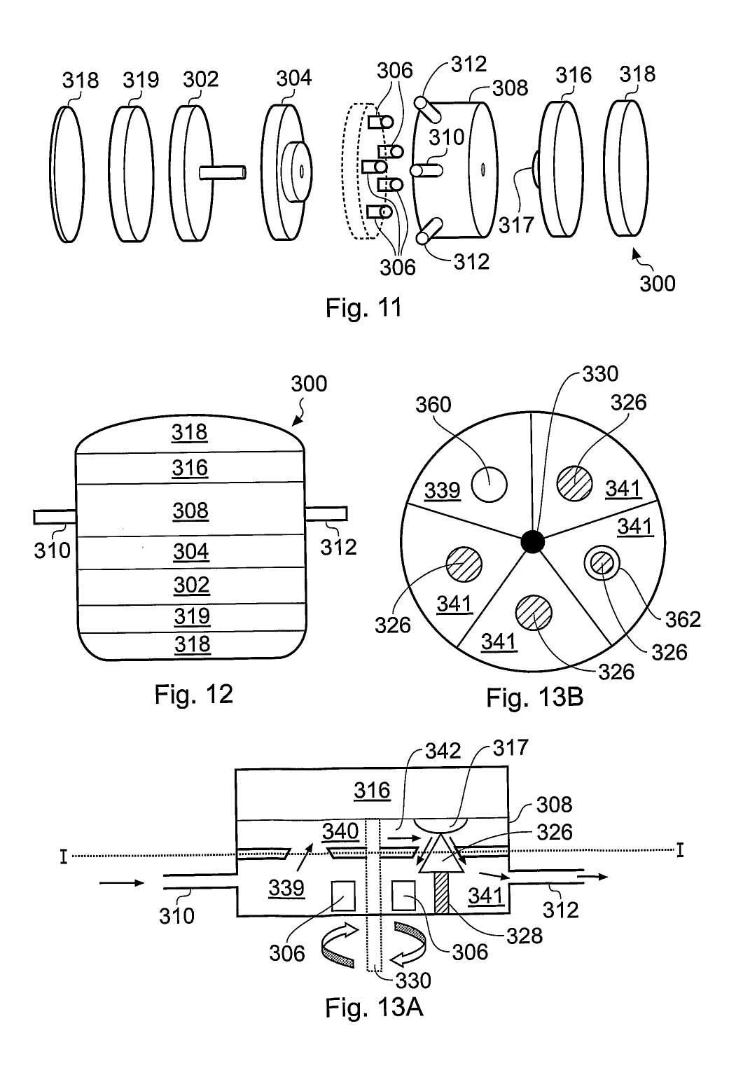

FIG. 11 is an exploded view of a fluid delivery device of the present invention,

FIG. 12 shows the device of FIG. 11 in an assembled state,

FIG. 13 illustrates the fluid flow pathway of the device of FIG. 11,

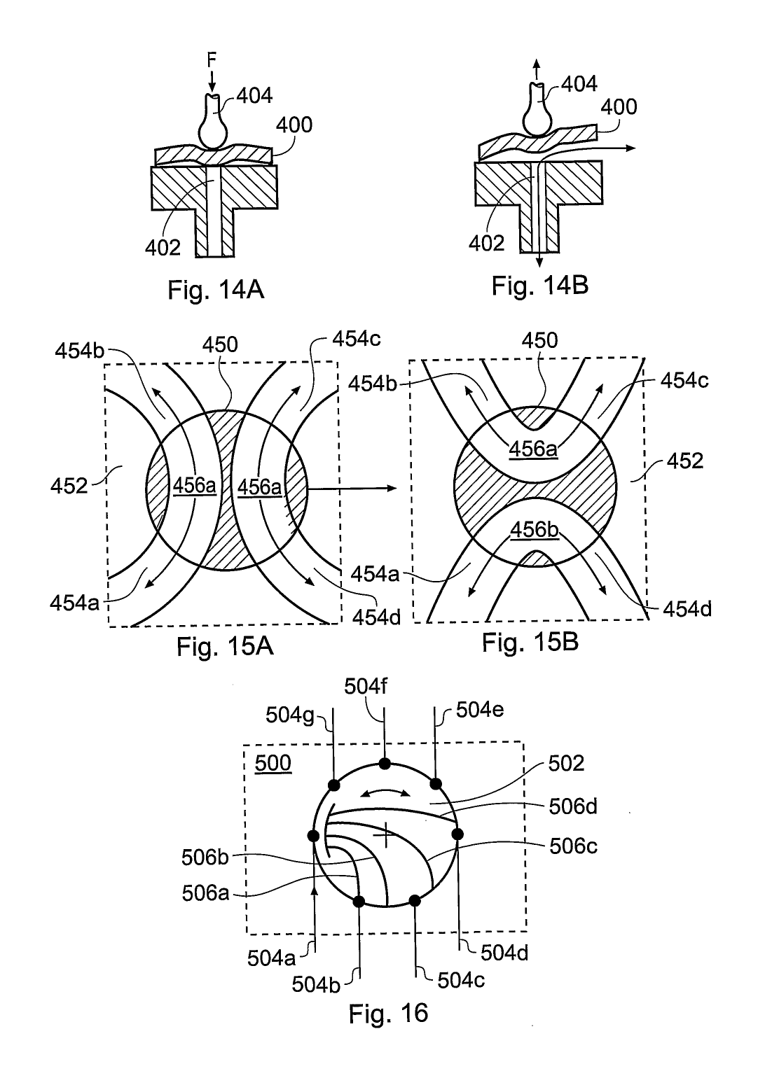

FIG. 14 shows a diaphragm version of the valve mechanism of FIG. 11-13,

FIG. 15 shows a further type of rotary valve mechanism,

FIG. 16 shows a Vernier type rotary valve mechanism, and

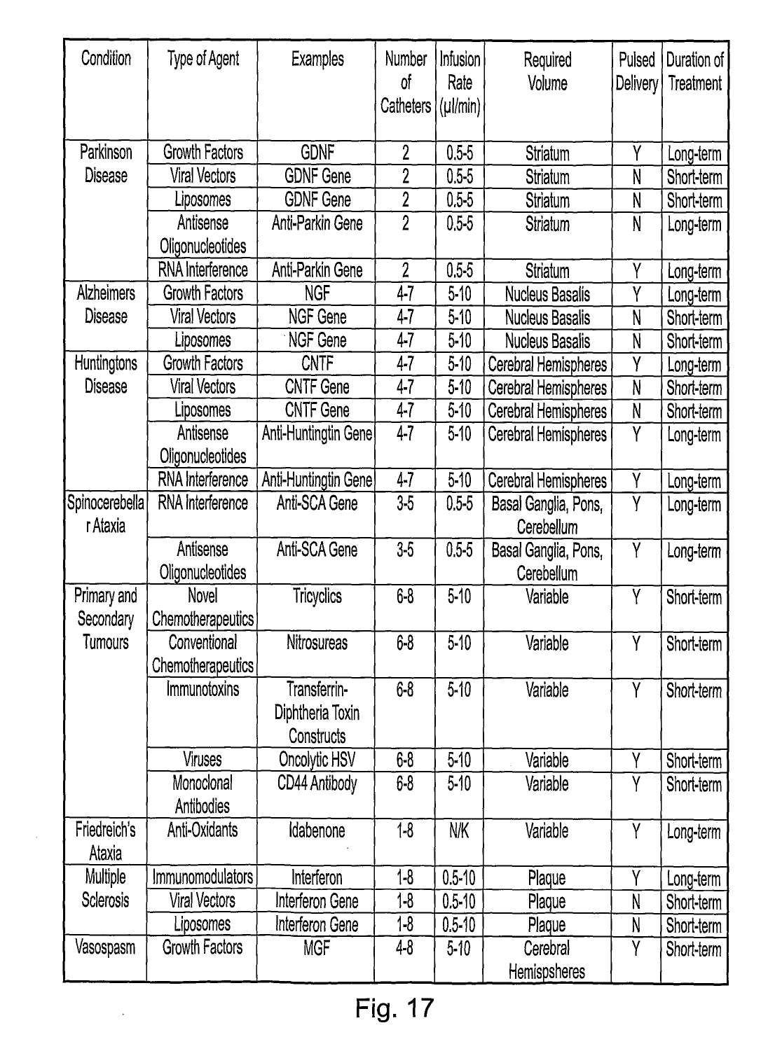

FIG. 17 lists various drug delivery applications of the present invention.

Referring to FIG. 1, a schematic view of a prior art implantable drug delivery system of the type described in WO2004/105839 is shown. A reservoir unit 21 is shown implanted subcutaneously over the anterior abdominal wall of a patient, and preferably within the rectus sheath anterior to the rectus muscle. The reservoir unit 21 has the purpose of holding a volume of a drug for infusion, and since the unit 21 is quite bulky in order to retain as much drug as possible, such a location is very suitable.

Leading from the reservoir unit 21 is a supply tube 22 which leads to a pump unit 23. The supply tube 22 is tunnelled subcutaneously between the reservoir unit 21 and the pump unit 23. The pump unit 23 is subcutaneously implanted in the subclavicular region. Implantation at this location is possible since the pump unit 23 is compact, made possible by the remote location of the reservoir unit 21. This location for the pump unit 23 is used as it should not prove to be inconvenient or uncomfortable to the patient, and yet it is close enough to the surface of the body that percutaneous refilling is relatively easy.

The pump unit 23 includes a refill port 24 on its front surface through which it is easy to palpate. The pump unit 23 includes one or more outlet ports 25 from which the drug is pumped into one or more outlet tubes. The outlet tubes 26 lead to intraparenchymal catheters 27 which are implanted in the brain of the patient.

Intraparenchymal catheters are known in the field of neurosurgery for delivering drugs to particular parts of the brain. The catheters are rigid tubes which are inserted stereotactically and secured to the skull with their distal ends in the vicinity of targets to be treated within the brain. The intraparenchymal catheters 27 are connected to the outlet tubes 26 which are tunnelled subcutaneously through the scalp and neck.

A prior art implantable drug delivery system of the type described in FIG. 1 allows a drug to be infused to multiple sites within the brain over prolonged periods of time. Although the relative volume of fluid supplied to different sites may be controlled by the tubing diameter used in the pump, the drug is supplied to each site simultaneously. It has, however, been found that such a drug delivery methodology does not always provide the optimum regimen. In particular the inventor has found that improved treatment can be obtained by sequentially (as opposed to simultaneously) delivering drugs to different parts of the brain.

Referring to FIG. 2, drug delivery apparatus of the present invention is shown that allows the sequential delivery of a drug to different regions of the brain. The apparatus comprises a implantable pump assembly 30 having an integral drug reservoir that is linked to the inlet of a skull mountable fluid distribution device 32 via a length of flexible tubing 34. The four outlets of the fluid distribution device 32 are respectively connected to four catheters 36a-36d (hereinafter collectively referred to as catheters 36) via four supply tubes 38a-38d (hereinafter collectively referred to supply tubes 38). The fluid distribution device 32, which is described in more detail below, is arranged to received fluid under pressure from the pump assembly 30 via tube 34 and to route the fluid to each of the catheters 36 in turn. Although rotating drug delivery to one catheter in turn is preferred, the fluid distribution device could be arranged to sequentially route fluid to exclusive subsets of catheters. For example, fluid may be routed to different catheter pairs in turn.

The entire drug delivery apparatus is implantable within a patient. For example, the pump assembly 30 may be implanted in the abdomen and the flexible tubing 34 tunnelled subcutaneously to the skull mounted fluid distribution device 32. Similarly, supply tubes 38 may be tunnelled subcutaneously through the scalp to each of the catheters 36. The catheters are tubes which are inserted stereotactically and secured to the skull with their distal ends in the vicinity of targets to be treated.

It should be noted that the four catheters 36 may be identical, or different, as required for the treatment of the particular medical condition. For example, a pair of minute catheters could be used to deliver a drug to the brainstem of a patient whilst a pair of larger catheters are used to supply the drug to the thalamus. Suitable catheters are described in WO2003/077785, the contents of which are incorporated herein by reference.

In certain circumstances, it is preferable for the drug delivery apparatus to provide convection enhanced drug delivery. In other words, the fluid pressure at the exit aperture of each catheter 36 may be arranged to be sufficient to overcome the turgor of the tissue but not so high as to cause back-flow of fluid along the catheter-tissue interface. In this manner, the drug can be driven deeper into the tissue than using diffusion drug delivery techniques. The regime in which convection enhanced delivery occurs will depend on the turgor of the tissue at each target site, the size of the catheter exit aperture and the pressure of the fluid at the catheter exit aperture. The skilled person would be able to arrange the apparatus to provide convection enhanced drug delivery if required.

Although the pump assembly 30 includes only a single drug reservoir, it would also be possible to use a pump assembly that draws fluid from multiple reservoirs. In such an arrangement, the pump assembly may be arranged to pass different drugs or drug concentrations to the single inlet of the fluid distribution device 32. Alternatively, a fluid distribution device could be provided with multiple inlets to separately receive different fluids via separate lengths of tubing or via multiple core tubing. In this latter case, the fluid distribution device could be arranged to mix drugs received from the two or more inlets and to selectively route such mixed fluids to an outlet. The relative mixing ratios of the fluids received from the two or more may be the same, or different, when directing the fluid to different outlets.

It should also be noted that although the examples contained herein describe delivering drugs to regions of the brain, the fluid distribution device could be used to deliver drugs, or any type of fluid, to any site(s) within the body. For example, drugs could be supplied to other parts of the central nervous system, major organs (e.g. a kidney, the liver etc) or muscles. Similarly, the skilled person would recognise that the device could be implanted in either a human or animal body as required.

Referring now to FIG. 3, a schematic illustration of the various components of the implantable fluid distribution device 32 described with reference to FIG. 2 is provided. The implantable fluid distribution device 32 comprises an inlet 40 for receiving fluid under pressure from the associated pump assembly 30 via flexible tubing 34. Fluid received at the inlet 40 is routed to a four way valve 42 via an optional pressure sensor 44 and/or an optional flow rate sensor 46. The four way valve 42 portion of the fluid distribution device may be implemented in a number of different ways as described in more detail below but the basic function of the valve 42 is to allow fluid received at the inlet 40 to be routed to any one of four outlets 50a-50d. The fluid output from the valve 42 may be routed to its associated outlet via optional fluid pressure sensors 52a-52d and/or optional flow rate sensors 54a-54d. Each outlet 50a-50d is connected to an associated catheter 36a-36d via tubing 38a-38d. A controller 56 is also provided to control operation of the valve 42 and to receive data from any pressure and/or flow rate sensors of the device. Although separate flow sensors may be provided, the skilled person would recognise that flow rate could be determined from the pressure drop between two points along a flow path. This would allows a pair of pressure sensors to be used to measure flow rate instead of providing separate flow rate sensors. One of such a pair of pressure sensors could be located at the remote pump if required.

In use, fluid is routed from the inlet 40 to each of the outlets 50a-50d in turn. Once implanted in a patient, a drug can thus be sequentially delivered to four sites within the brain via the four catheters 36. The sequential (rather than simultaneous) delivery of drugs to different target sites within the brain has been found to greatly improve the effectiveness of, and/or reduce side effects associated with, certain treatments. For example, the sequential delivery of chemotherapy drugs to sites adjacent a brain tumour has been found to have a lower detrimental impact on the patient than simultaneously delivering such drugs to the same sites. It should also be noted that the four way valve 42 may have a fifth “off” state in which fluid flow to all outlets is prevented. In other words, the sequential delivery of drugs via the different catheters may be interspersed with periods in which no drug is delivered.

Operation of the fluid distribution device 32 is controlled by the controller 56. In the simplest configuration, the controller 56 is arranged (e.g. pre-programmed) to actuate the valve 42 so as to sequentially route fluid to each outlet for a predetermined duration. In such an arrangement, fluid may be routed to each outlet 50 for the same duration or fluid may be routed to different outlets for different periods of time. The controller 56 may also be arranged such that there are periods of time in which all fluid flow is prevented.

The pre-programmed routing schedule of the fluid distribution device 32 may be synchronised with the fluid pressure and/or the fluid composition that is provided by the pump assembly 30. For example, the pump may be arranged to deliver fluid at a first pressure during periods in which the fluid distribution device is routing fluid to a first catheter and at a second pressure during periods when the fluid distribution device is routing fluid to a second catheter. The pump assembly 30 may also be arranged to stop fluid flow during periods when the fluid distribution device 32 is in an “off” state in which fluid flow to all outlets is prevented.

The controller 56 may also be configured to monitor the pressure of fluid received at the inlet 40 using the optional pressure sensor 44. Alternatively, or additionally, the controller 56 may be arranged to monitor the amount of fluid passing into the fluid distribution device 32 using the optional flow rate sensor 46. Such an arrangement allows the measured input fluid pressure and/or the input fluid flow rate to be used by the controller 56 to determine the required duration of drug delivery via the different catheters. The controller 56 may also, or alternatively, be configured to monitor the fluid pressure and/or fluid flow rate at each outlet using the optional pressure sensors 52 and/or the optional flow rate sensors 54. In this manner, the amount of fluid and/or the pressure of fluid output by the device can be measured. Such measurements may be used by the controller 56 to calculate the duration of fluid delivery that is required via each catheter 36 to provide, for example, a certain drug dosage.

The fluid distribution device 32 may also be arranged so that the pressure of the fluid at the selected outlet 50 is lower than the pressure of the fluid received at the inlet 40. For example, valve 42 may act not only as a router but may also be arranged to provide some control over the fluid pressure and/or flow rate to the outlets 50. Alternatively, a separate pressure regulator (not shown) may be provided so that the routing valve 42 receives fluid at a lower pressure than the pressure at the inlet 40. Fluid pressure regulation may be predetermined (e.g. a fixed pressure regulator may be provided) or the fluid pressure at each outlet may be dynamically controlled by the controller 56. Providing fluid pressure regulation may be advantageous where the pressure of fluid received at the inlet 40 varies over time but a constant pressure of fluid at each outlet is required.

The controller 56 may be configured (e.g. programmed) to provide a certain drug delivery regimen prior to implantation of the fluid distribution device 32 in a patient. The fluid distribution device 32 could also comprise a communications device (e.g. a RF transmitter/receiver) to allow a telemetry link to be established after implantation. In this manner, data can be received from, and/or sent to, the device when it is implanted in a patient. The telemetry link may also be used to receive information from an implanted device enabling, for example, information to be received from the device about the amount of drug that has been delivered, the flow rate through the various catheters etc. Such data may be used to ensure correct operation of the drug delivery system after implantation. The telemetry link may also be used to send commands to the controller 56 that allows the drug delivery programme to be altered as required.

The pump assembly 30 for supplying fluid to the inlet 40 of the fluid distribution device 32 may be of any known type. For example, the pump 30 assembly could be a constant pressure pump. Constant pressure pumps typically comprise a drug reservoir located within a housing that contains a gas such that, when the reservoir is filled, the gas is compressed which in turn provides the pressure to empty the reservoir. In other words, the energy to required to expel the fluid under pressure is providing by the filling process and no separate power source is required. Alternatively, an electrically powered pump assembly may be provided.

Referring to FIG. 4, the outer housing of an implantable fluid distribution device 32 of the type described above with reference to FIGS. 2 and 3 is shown. The fluid distribution device 32 is substantially cylindrical and has a domed upper surface 64. The fluid distribution device 32 has an upper portion 66 which contains the valve 42 and any necessary tubing etc and a lower portion 68 that contains the electronics of the controller 56 and the batteries required for powering the device. The majority of the lower portion 68 of the device can be located within a recess or hole formed in the skull 70 of a patient. Protruding flanges 72 allow the device 32 to be securely attached to the skull using screws 74. The inlet 40 and outlets 50 (noting that only outlets 50a and 50b are shown in FIG. 4) of the device are distributed around the radius of the upper portion 66 and protrude substantially perpendicularly therefrom. The inlet 40 and each of the outlets 50 comprise a nozzle to which tubing (e.g. flexible tubing 34 and catheter supply tubes 38) can be securely attached using sutures. Alternatively, the nozzles could comprise a barbed end to which tubing could be attached or a “snap-fit” connector.

The fluid distribution device 32 described with reference to FIGS. 2 to 4 houses all the necessary control electronics and batteries. However, it is also possible to locate the power source and/or some of the electronics remotely to the fluid distribution device. An example of such a drug delivery apparatus will now be described with reference to FIG. 5.

The apparatus shown in FIG. 5 comprises a pump assembly 130 attached to a remote fluid reservoir 128 via a length of flexible plastic tubing 126. A fluid distribution device 132, suitable for mounting on the skull of a patient, is also shown. The fluid distribution device 132 has an inlet for receiving fluid from the pump assembly 130 via flexible tubing 134. The fluid distribution device 132 is also connected to catheters 36a-36d respectively (hereinafter collectively referred to as catheters 36) via four associated supply tubes 38a-38d (hereinafter collectively referred to supply tubes 38).

The fluid distribution device 132 performs a similar function to the fluid distribution device 32 described with reference to FIGS. 2 to 4 above. However, the fluid distribution device 132 does not comprise an integral power source. Instead, the flexible tubing 134 has an insulated electrical cable 135 wrapped around or formed within its outer surface. A power source (e.g. a battery) is located within the pump assembly 130 and power is supplied to the fluid distribution device 132 via the electrical cable 135. The pump assembly 130 may comprise an electrical pump which could also be powered by the same power source that is used to power the fluid distribution device 132. Locating the batteries within the pump assembly 130 reduces the size of the fluid distribution device 132 relative to the fluid distribution device 32 described with reference to FIGS. 2 to 4 above.

In addition to a remote power source, some or all of the electronics used to control operation of the fluid distribution device 132 may be located within the pump assembly 130. In such an example, a multiple core electrical cable could be used to send both power and control signals from the pump assembly to the fluid distribution device 132. Alternatively, the fluid distribution device 132 could be controlled by electronics within the pump assembly 130 over a wireless link. In such an arrangement, the fluid routing function of the fluid distribution device 132 can be synchronised with pump control allowing, for example, different fluid pressures or drug compositions output by the pump to be routed to different catheters.

Referring to FIG. 6, a valve assembly portion 150 suitable for inclusion in a fluid distribution device of the type described above is illustrated. The valve 150 comprises an input tube which branches into four output tubes 152a-152d and also includes a rotatable member 154 having four segments 156a-156d. Although not shown, a motor may also be provided (e.g. an electrical stepper motor) to rotate the rotatable member 154.

Each of the four segments 156a-156d of the rotatable member 154 is located adjacent one of the four deformable tubes 152a-152d and has a cross-sectional profile comprising a circular portion and a substantially flat portion. Rotation of the rotatable member 154 allows the flat or circular cross-sectional portions of each segment to be located adjacent the associated flexible tube as required. The circular cross-section portion of each segment is arranged to deform the associated flexible tube thereby preventing fluid flow through that tube and the substantially flat cross-section of the segment is arranged to impart minimal tube deformation thereby allowing fluid to pass through the tube. The flat portions of each segment are offset so that, at any one time, fluid may pass through only one of the four output tubes 152a-152d. Rotation of the rotatable member 154 allows the one tube which is “open” to be selected as required. Rotation of the rotatable member 154 may also be used to control the amount of tube deformation thus providing control over the flow rate through the “open” tube.

Referring to FIG. 7, an alternative valve assembly 170 suitable for inclusion in a fluid distribution device of the type described above is illustrated. The assembly comprises an outer housing 172 having multiple outlets 174. Contained within the outer housing 172 is a rotatable member 178 having an aperture 180 and an inlet 182 to receive fluid (e.g. from a remote pump). A fluid seal is provided such that any fluid entering the rotatable member 178 from the inlet 182 can only exit the rotatable member 178 through aperture 180. Rotation of the rotatable member 178 allows the aperture to be aligned with any one of the outlets 174 allowing fluid to be selectively routed from the inlet 182 to any one of the outlets 174.

Referring to FIG. 8, a further valve assembly 190 suitable for inclusion in a fluid distribution device of the type described above is illustrated. The valve assembly 190 comprises a substantially cylindrical outer housing 191 having a plurality of fluid outlets 192. A substantially cylindrical inner member 194 having a plurality of apertures 196 is retained within the outer housing 191. The inner member 194 also comprises an inlet 198 for receiving fluid under pressure. The inner member 194 is rotatable within the outer housing 191 and a fluid seal between the inner member 194 and outer housing 191 is provided such that fluid received from the inlet 198 can only pass through an aperture 196 when that aperture is aligned with its associated fluid outlet 192. In this manner, fluid communication can be selectively established between the inlet 198 and any one of the outlets 192 by rotation of the inner member 194 within the outer housing 191.

Referring to FIG. 9, a further valve assembly 200 suitable for inclusion in a fluid distribution device of the type described above is illustrated. The valve assembly 200 comprises a substantially cylindrical outer housing 202 having a plurality of radially offset fluid outlets 204. A substantially cylindrical inner member 206 having a plurality of apertures 208 is retained within the outer housing 202. The inner member 206 also comprises an inlet 210 for receiving fluid under pressure and is axially translatable relative to the outer housing 202. A fluidic seal is provided between the inner member 206 and outer housing 202 such that fluid can only pass through an aperture when such aperture is aligned with its associated fluid outlet 208. Fluid communication can thus be selectively established between the inlet 210 and any one of the outlets 204 by linear translation of the inner member 206 within the outer housing 202.

A single fluid distribution device (e.g. fluid distribution device 32 or 132 described with reference to FIGS. 2 to 9 above) could be implanted in a patient, or a number of such devices could be implanted and linked together. Two ways in which fluid distribution devices could be linked together are illustrated in FIG. 10.

Referring to FIG. 10a, fluid delivery apparatus is shown that comprises a first fluid distribution device 230 and a second fluid distribution device 232. The first fluid distribution device 230 comprises an inlet 234 and three outlets 236. The second fluid distribution device 232 comprises an inlet 238 and two outlets 240. The inlet 238 of the second fluid distribution device 232 is connected to an outlet 236 of the first fluid distribution device 230. Catheters can then be connected to the other outlets 236 of the first fluid distribution device 230 and the outlets 240 of the second fluid distribution device 232. This arrangement thus allows fluid to be routed to any one of four catheters.

Referring to FIG. 10b, fluid delivery apparatus is shown that comprises two fluid distribution devices 240. Each fluid distribution devices 240 comprises an inlet 242 and a pair of outlets 244. A branched tube 246 is arranged to route fluid from a remote pump assembly (not shown) to the inlet of each of the fluid distribution devices 240. This arrangement also allows fluid to be routed to any one of four catheters connected to the four outlets 244.

If two or more fluid distribution devices are provided, coordinated fluid routing operation could be implemented. For example, the fluid distribution devices could be programmed to route fluid to certain outlets for certain intervals of time. In this case, operation of the two fluid distribution devices could synchronised before implantation. Alternatively, some kind of data link 248 could be provided between the two fluid distribution devices. In this case, one fluid distribution device could be a master unit and the other device could be a slave unit. Alternatively, the two or more fluid distribution devices could be cabled to a remote control unit; for example an associated pump assembly may comprise the power source and/or control electronics necessary for distribution device operation. Although the combination of two fluid distribution devices is shown, the skilled person would recognise that any number of units could be linked together as required.

Referring to FIGS. 11 to 13, an alternative design of fluid distribution device 300 of the present invention is illustrated. In particular, FIG. 11 gives an exploded view of the various components of the fluid distribution device 300 that is shown in its assembled state in FIG. 12.

Referring to FIG. 11, the fluid distribution device 300 comprises a stepper motor 302, control electronics 304, five pressure sensors 306, a fluidic housing 308 and a annular, rotatable, portion 316 having a protruding feature 317. End caps 318 and a battery 319 are also provided. The fluidic housing 308 comprises an inlet 310 and four outlets 312 (noting that only two of the outlets are shown in FIG. 11). The fluidic housing 308 comprises a fluid pathway that runs from the inlet 310 to a central internal cavity. Each of four further cavities are in fluid communication with an associated outlet 312. Each further cavity is also coupled to the central internal cavity via an aperture and a flow control member is provided in the vicinity of each aperture.

Each flow control member is spring loaded so that it engages, and provides a fluid seal with, its associated aperture in the absence of an externally applied force. The protrusion 317 of the rotatable portion 316 is arranged so that it can be urged against any selected flow control member thereby causing that flow member to disengage its aperture and allowing the passage of fluid to the associated outlet. In this manner, the amount of fluid flow from the central internal cavity to the selected outlet can be controlled by varying the force that is applied to the flow control member by the protrusion 317 of the rotatable portion 316. The five pressure sensors 306 allow the inlet pressure and the pressure at each of the four outlets to be separately monitored.

The stepper motor 302, the operation of which is controlled by the control electronics 304, has a shaft which passes through apertures formed in the control electronics 304 and fluidic housing 308 portions and engages the rotatable portion 316. Rotation of the rotatable portion 316 by activating the stepper motor 302 allows the protrusion 317 of the rotatable portion 316 to be aligned with any selected one of the flow control members. The protrusion 317 has a ramped surface thus also providing flow control. It can thus be seen that rotating the rotatable portion 316 using the stepper motor 302 allow fluid to flow at a required flow rate from the inlet to any one selected outlet.

Referring to FIG. 12, the fluid distribution device 300 described with reference to FIG. 11 is shown in its assembled state. The device 300 comprise the stepper motor 302, electronics 304 and fluidic housing 308 plus a battery 319. As described above with reference to FIG. 4, the device is suitable for implantation in a recess formed in, or hole through, the skull.

Referring to FIGS. 13a and 13b, a more detailed illustration of the fluidic housing 308 described with reference to FIGS. 11 and 12 is shown.

FIG. 13a shows the housing 308 which comprises a central internal cavity 340 that is in fluid communication with an inlet 310 via an inlet cavity 339. Apertures are formed in the walls of the central internal cavity 340 through which fluid can pass to further outlet cavities 341. Each outlet cavity 341 is also in fluid communication with an associated outlet 312. A conical plug 326 and a spring loaded mounting 328 are also provided in each outlet cavity 341. In the absence of any additional force, the spring loaded mounting 328 is arranged to force the conical plug 326 into engagement with the walls defining the aperture. The rotatable portion 316 is linked to the shaft 330 of the stepper motor 302. Pressure sensors 306 are also provided in the inlet cavity 339 and the outlet cavities 341. Rotation of the rotatable portion 316 allows the protrusion formed therein to be aligned with any one of the plugs 326 thereby causing that plug to disengage its associated aperture thereby allowing fluid flow therethrough. Providing a suitably shaped protrusion (e.g. a ramp) allows the position of the associated rubberised plug 326 relative to the aperture to be controlled. In other words, the flow rate through the aperture may be controlled by varying the position of the rubberised plug using the rotatable portion 316 so as to partially block the aperture as required.

FIG. 13b shows a cross-sectional view of the housing along the line I-I of FIG. 13a. It can be seen that the housing 308 is divided into five segments defining the four outlet cavities 341 and the one inlet cavity 339. The aperture 360 of the inlet cavity 341 is unobstructed. The apertures of three of the four outlet cavities are blocked by the associated plug 326. The aperture 362 of one outlet cavity is not sealed by the associated plug 326; i.e. in this configuration the plug 326 has been forced out of engagement with the aperture 362 by the protrusion 317 of the rotatable portion 317 as shown in FIG. 13a.

It should be noted that the illustrations of FIGS. 11 to 13 are schematic only and the skilled person would appreciate that many variations to the basic concept would be possible. For example, the housing may be designed to minimise dead-space in the fluid pathway. A similar arrangement could also be provided in which the rotatable portion 316 and spring loaded mechanism are replaced with separate piezo-electric actuators for urging a plug into each aperture; such actuators may be bistable to minimise power consumption. Furthermore, the plugs may be spring loaded such that they disengage each associated aperture in the absence of an applied force. In such a case, the rotatable portion 316 could comprise a recess so that the physical force applied to the plug is released when the recess is aligned with a plug.

FIG. 14 illustrates a variant of the device shown in FIGS. 11-13. Instead of providing a conical plug 326 for selectively blocking each aperture as shown in FIG. 13a, FIG. 14 illustrates a device in which a flexible diaphragm 400 is used to cover an aperture 402. A bistable actuator 404 is also provided that can be urged into contact with the diaphragm. FIG. 14a shows the “closed” state in which the actuator 404 forces the diaphragm 400 into contact with the aperture 402 thereby preventing any fluid flow therethrough. FIG. 14b shows the “open” state in which the diaphragm is not pushed against the aperture thereby permitting fluid to pass. Intermediate flow states could also be provided if required. Although only one aperture is shown in FIG. 14, a single diaphragm could cover a plurality of apertures. Individual actuators could then be provided for forcing the diaphragm into contact with each actuator, or a single actuation mechanism analogous to the rotatable portion 316 described above could be used for controlling flow through all apertures.

FIG. 15 illustrates a further rotary valve mechanism. A central portion 450 is provided that is rotatable relative to a base portion 452. The base portion 452 comprises four channels 454a-454d connected to the inlet/outlet ports of the mechanism as required. The central portion 450 comprises two channels 456a and 456b. In the rotational position shown in FIG. 15a, channels 454a and 454b of the base portion 452 are in fluid communication via channel 456a of the central portion 450 and channels 454c and 454d of the base portion 452 are in fluid communication via channel 456b of the central portion 450.

Rotating the central portion through about 90° as shown in FIG. 15b alters the fluid pathway through the valve. In particular, it can be seen from FIG. 15b that such a rotation causes channels 454b and 454c of the base portion 452 to be in fluid communication via channel 456a of the central portion 450 and channels 454a and 454d of the base portion 452 to be in fluid communication via channel 456b of the central portion 450. If channel 454a is in fluid communication with an inlet, it can be seen that rotation of the central portion 450 allows fluid to be routed to either one of channels 454b or 454d which in turn can be in fluid communication with respective outlets. Also, fluid can be selectively routed from channel 454c to either of 454b or 454d. Although a four port valve is shown, more or fewer ports may be provided as required.

FIG. 16 illustrates a “Vernier” version of the rotary valve mechanism of FIG. 15 in which reduced angular rotation is required to alter the fluid routing path through the device. The valve mechanism comprises a base portion 500 and a rotatable portion 502. The base portion comprises a plurality of channels 504a-504g that terminate at the rotatable portion 502. The rotatable portion comprises four channel 506a-506d. The proximal end of each channel 506 of the rotatable portion 502 are in fluid communication with one another and the distal ends of these channels are distributed around the periphery of the rotatable portion 502.

In use, the proximal ends of the channels 506 of the rotatable portion 502 are placed in fluid communication with a selected channel of the base portion 500. FIG. 16 shows channel 504a of the base portion 500 in fluid communication with the proximal ends of channels 506a-d of the rotatable portion 502. FIG. 16 also shows the distal end of channel 506a of the rotatable portion 502 aligned with channel 504b of the base portion 500; this configuration allows fluid to pass from channel 504a to channel 504b via the rotatable portion 502. A small anticlockwise rotation of the rotatable portion 502 will cause fluid communication between channels 506a and 504b to be broken, but fluid communication will then be established between channel 506b and 504c. Further anti-clockwise rotation then establishes fluidic connection between channels 506c and 504d etc. In this manner, relatively small rotational movements of the rotatable portion 502 can be used to route fluid to different outlets. Larger movements may also be used to alter the channel of the base portion 500 through which fluid can pass to the proximal end of each channel 506 of the rotatable portion 502. In other words, the valve mechanism may provide a plurality of inlets. The apparatus described herein can be implanted so as to deliver a fluid to any site or sites within a human or animal body. The apparatus is, however, particularly suited for use in medical treatments that involve supplying some kind of therapeutic agent/drug to the brain via one or more implanted catheters. The fluid may comprise an agent or agents dissolved therein or it may comprise particles (e.g. gene therapy agents, nano-particles, viral vectors, liposomes etc) carried by the fluid.

Referring to FIG. 17, a number of potential applications for convection enhanced delivery to the brain are provided. In particular, the type of agent and the number of 0.2 mm outer diameter catheters required to deliver such an agent are shown. In certain circumstances, the delivery regimen may require continuous delivery whilst other treatments may require pulsed (bolus) delivery. The sequential delivery of therapeutic agents through catheters (or sets of catheters) in turn may also provides improved treatment efficacy. It should be noted that the list of FIG. 17 is by no means exhaustive. The skilled person would appreciate the numerous applications in which apparatus of the type described above could be used.

1. An implantable fluid distribution device comprising;
a first inlet for receiving fluid under pressure from a remote pump;
a plurality of outlets, each outlet being connectable to a fluid delivery catheter; and
a valve mechanism for controlling the passage of fluid from the first inlet to the plurality of outlets.

2. A device according to claim 1 wherein the valve mechanism controls at least one of the flow rate and pressure of fluid passing therethrough.

3. A device according to claim 1 wherein the valve mechanism provides a shut-off state in which no fluid can pass from the first inlet to any of the plurality of outlets.

4. A device according to claim 1 comprising two or more mutually exclusive sets of outlets, each set of outlets comprising at least one of said plurality of outlets, wherein the valve mechanism permits fluid to pass from the first inlet to no more than one set of outlets at a single instant in time.

5. A device according to claim 1 comprising a controller for controlling operation of the valve mechanism.

6. A device according to claim 5 comprising two or more mutually exclusive sets of outlets, each set of outlets comprising at least one of said plurality of outlets, wherein the controller is arranged to operate the valve mechanism such that fluid is passed from the first inlet to each set of outlets in sequence.

7. A device according to claim 1 comprising at least one sensor for sensing at least one of fluid pressure and fluid flow rate.

8. A device according to claim 7 wherein said at least one sensor is located within the valve mechanism.

9. A device according to claim 1 comprising at least one additional inlet, wherein the valve mechanism also controls the passage of fluid from the at least one additional inlet to the plurality of outlets.

10. A device according to claim 9 wherein the valve mechanism comprises a mixing chamber for receiving fluid from the first inlet and from the at least one additional inlet.

11. A device according to claim 1 comprising at least three outlets.

12. A device according to claim 1 comprising at least four outlets.

13. A device according to claim 1 in which the valve mechanism comprises a chamber, wherein the chamber is in fluid communication with the first inlet and has a plurality of output apertures, wherein any fluid exiting the chamber via an output aperture is routed to one of said plurality of outlets.

14. A device according to claim 13 in which the valve mechanism comprises a moveable plug associated with each output aperture, wherein movement of each moveable plug controls fluid flow through the associated aperture.

15. A device according to claim 14 wherein the valve mechanism comprises a plurality of electrically powered actuators, each electrically power actuator being arranged to move at least one of said moveable plugs.

16. A device according to claim 14 wherein the valve mechanism comprises a single moveable member that can urge each of said plurality of moveable plugs into contact with its associated aperture.

17. A device according to claim 1 wherein the valve mechanism comprises an inner member substantially retained within, and moveable relative to, an outer member.

18. A device according to claim 17 wherein the inner member defines a cavity in fluid communication with said first inlet and the outer member comprises a plurality of output ports, each output port being in fluid communication with one of said outlets, wherein the inner member has at least one aperture formed therein and movement of the inner member relative to the outer member permits fluid to be selectively routed from the first inlet to any one or more of said plurality of outlets through the at least one aperture of the moveable member.

19. A device according to claim 18 wherein the inner member is at least one of axially translatable and rotatable relative to the outer member.

20. A device according to claim 1 in which the valve mechanism comprises a first length of resiliently deformable tubing for receiving fluid from the first inlet and one or more additional lengths of resiliently deformable tubing for delivering fluid to the plurality of outlets, wherein the valve mechanism comprises a single flow control member that can be selectively urged against each of the one or more additional lengths of resiliently deformable tubing thereby controlling the passage of fluid therethrough.

21. A device according to claim 20 wherein the valve mechanism comprises an electrically powered actuator for moving said flow control member.

22. A device according to claim 1 comprising an integral electrical power source.

23. A device according to claim 1 wherein the first inlet and each of the plurality of outlets comprise a connector for connecting to a tube.

24. A device according to claim 1 wherein substantially all of the fluid contacting surfaces of the valve mechanism are formed from a material that does not significantly reduce viral activity.

25. A device according to claim 24 wherein said fluid contacting surfaces comprise at least one of polypropylene, High Density Polyethylene (HDPE), Polytetrafluoroethylene (PTFE), Ethylene/Tetrafluoroethylene Copolymer (ETFE), Flouroethylene-propylene (FEP), Polyethylene Terephthalate (PET), polyurethane, glass and ceramic.

26. A device according to claim 1 that is configured to be mounted subcutaneously.

27. A device according to claim 26 that is configured to be mounted, at least in part, in a recess formed in the skull.

28. A device according to claim 1 comprises an outer, substantially cylindrical, housing.

29. Drug delivery apparatus comprising an implantable fluid distribution device according to claim 1 and at least one pump assembly, wherein flexible tubing is provided to carry fluid from the at least one pump assembly to the fluid distribution device.

30. An apparatus according to claim 29 wherein a single controller is provided to control operation of the pump and the fluid distribution device.

31. Drug delivery apparatus comprising an implantable fluid distribution device according to claim 1 and a plurality of fluid delivery catheters, wherein each outlet of the fluid distribution device is connected to a fluid delivery catheter.

32. An apparatus according to claim 31 wherein each outlet is connected to a fluid delivery catheter via a length of flexible tubing.

33. An apparatus according to claim 29 comprising; one or more sensors for sensing the pressure of fluid at one or more locations within said apparatus; and a controller for controlling operation of the valve mechanism of the fluid distribution device, wherein the controller is arranged to ensure the fluid pressure within the apparatus does not exceed a predetermined value.

34. A method of surgery comprising the step of implanting at least one of a device according to claim 1 and a drug delivery apparatus including the device and at least one pump assembly, wherein flexible tubing is provided to carry fluid from the at least one pump assembly to the fluid distribution device.

35. A method for delivering drugs to the brain comprising the steps of: (a) taking a subject brain having a plurality of catheters implanted therein, and (b) sequentially delivering a fluid to two or more mutually exclusive subsets of said catheters.

36. A method according to claim 35 wherein step (a) comprises implanting a plurality of catheters into the subject brain.

37. A method according to claim 36 wherein step (a) comprises implanting said plurality of catheters in locations that allow a drug to be delivered to substantially the whole brain.

38. A method according to claim 35 wherein step (b) comprises determining the amount of fluid delivered to each catheter.

39. A method according to claim 35 wherein step (b) comprises selecting the amount of fluid delivered to each catheter.

40. A method according to claim 35 wherein step (b) is repeated a plurality of times and the relative amount of fluid delivered by each catheter is altered between at least two subsequent repetitions of said step.

41. A method according to claim 35 wherein step (b) comprises the step of delivering a drug.

42. A method according to claim 35 wherein step (b) comprises delivering a fluid via convection enhanced delivery.
